(19) United States
(12) Patent Application Publication
(10) Pub. No.: US 2012/0040939 A1
(43) Pub. Date: Feb. 16, 2012

(54) MEDICINAL USE OF 5-BENZYLAMINOSALICYLIC ACID DERIVATIVE OR ITS SALT

(75) Inventors: Byoung-Joo Gwag, Suwon Gyeonggi-Do (KR); Sun-Joo Son, Suwon Gyeonggi-Do (KR); Jae-Sung Noh, Anyang Gyeonggi-Do (KR); Jin-Hee Shin, Seoul (KR)

(73) Assignee: NEUROTECH PHARMACEUTICALS CO., LTD., Yeongtong-Gu (KR)

(21) Appl. No.: 13/148,414

(22) PCT Filed: Feb. 9, 2010
(86) PCT No.: PCT/KR2010/000783

§ 371 Date: Nov. 4, 2011

(30) Foreign Application Priority Data
Feb. 9, 2009 (KR) 10-2009-0010068
Feb. 8, 2010 (KR) 10-2010-0011521

(51) Int. Cl. A61K 31/606; A61K 31/616; A61P 25/22; A61P 25/24; A61P 25/00; C07C 229/64; A61K 31/618

(52) U.S. Cl. 514/166; 562/453; 560/138; 560/46

(57) ABSTRACT

The present invention relates to a pharmaceutical composition for treating or preventing stress disorder, depression, anxiety disorder, comprising 5-benzylaminosalicylic acid derivative or its pharmaceutically acceptable salt. The present invention relates to a method for treating or preventing stress disorder, depression, anxiety disorder, comprising 5-benzylaminosalicylic acid derivative or its pharmaceutically acceptable salt.

### TECHNICAL FIELD

The present invention relates to a pharmaceutical composition for treating or preventing stress disorder, depression, anxiety disorder, comprising 5-benzylaminosalicylic acid derivative or its pharmaceutically acceptable salt. The present invention relates to a method for treating or preventing stress disorder, depression, anxiety disorder, comprising 5-benzylaminosalicylic acid derivative or its pharmaceutically acceptable salt.

### BACKGROUND ART

Stress occurs when a person can not respond appropriately to emotional or physical threats and is often accompanied by symptoms such as tissue damage, hemorrhage, infection, hypoglycemia, pain, etc. In addition, excessive stress is the cause of mental illness such as depression or anxiety disorders etc.

Depression, a stress-related mental disease, recurs frequently, tends to be chronic, and causes serious consequences such as suicide. Main symptoms of depression are depressed mood and emotion and appear along with resultant loss of sleep, appetite, and interest, and anxiety, helplessness, guilt, isolation, and futility, impulsivity towards suicide etc. Change in weight is severe and the behavior becomes very dull and slow. And, depression is accompanied by feelings of worthlessness, inappropriate guilt, and decreased concentration and memory. The depression patients feel chronically tired and can not sleep in many cases and have a bad night's sleep. In addition to changes in emotions, thoughts, and desires, physical symptoms such as headache, indigestion, stiff neck and shoulders, chest tightness and so on appear. In case of severe depression, delusions or hallucinations might occur.

It is known that depression is caused by not only by social and environmental factors such as several psychological factors, shock or stress etc. but also by biological factors such as disorders of nervous system or endocrine system.

Drug intakes such as antihypertensive drugs, anxiolytics, psychotropic drugs, and central nervous system stimulants etc. can be a cause of depression. Diabetes, pancreatic cancer, or endocrine disease also can be a cause of depression.

Since the 1950s, tricyclic antidepressants (TCAs) began to be used as an effective first-line therapy for depression through the mechanism increasing concentration of serotonin and epinephrine in the synapse. Although the efficacy of TCAs is very good, central nervous system side effects such as sedation, confusion, delusions, compulsive disorder, headaches, sleep disturbances and cardiovascular side effects such as postural hypotension and tachycardia, and anticholinergic side-effects remain to be considerable problems. After that, since 1980s, launching of selective serotonin reuptake inhibitors (SSRIs) which have similar effects but superior in terms of side effects and safety compared with TCAs has brought great changes in the therapy of depression. Currently, fluoxetine, citalopram, sertraline, paroxetine, and escitalopram are commercially available, occupying about 70% of antidepressants.

However, these SSRIs also show headache, nausea, appetite inhibition, sexual dysfunction, sleep disturbances, fatigue, weight change, suicidal ideation and extrapyramidal side effects. Recently, antidepressant drugs with compound pharmacological actions have been used to enhance therapeutic effects including serotonin and norepinephrine reuptake inhibitors (SNRIs) such as venlafaxine and milnacipran, a noradrenergic and a specific serotonergic antidepressant (NSSA), mirtazapine, and norepinephrine and dopamine reuptake inhibitors (NDRIs), bupropion etc.

Clinical symptoms and medications of major depression tend to overlap with anxiety disorders including panic-agoraphobia syndrome, severe phobias, generalized anxiety disorder (GAD), social anxiety disorder, post-traumatic stress disorder (PTSD), and obsessive-compulsive disorder (OCD) etc. In fact, antidepressants such as tricyclic antidepressants and serotonin reuptake inhibitors are used in the treatment for panic disorder, agoraphobia, obsessive-compulsive and traumatic stress disorder.

### DISCLOSURE

### Technical Problem

Accordingly, the object of the present invention is to provide a pharmaceutical composition useful for treating or preventing stress disorder, depression, anxiety disorder, and a method for treating or preventing stress disorder, depression, or anxiety disorder.

### Technical Solution

To achieve the object, the present invention provides a pharmaceutical composition for treating or preventing stress disorder, depression or anxiety disorder, comprising 5-benzylaminosalicylic acid derivatives represented by the below chemical formula 1 or its pharmaceutically acceptable salts as effective agents:

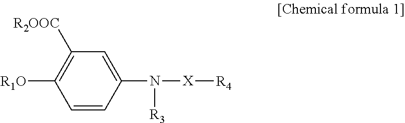

wherein,
X is CO, SO₂ or (CH₂)ₙ (where n is an integer of 1 to 5, inclusive);
R₁ is hydrogen, alkyl or alkanoyl;
R₂ is hydrogen or alkyl;
R₃ is hydrogen or an acetyl group; and
R₄ is phenyl group which is unsubstituted or substituted with one or more of the group consisting of nitro, halogen, haloalkyl, and C₁-C₅ alkoxy; or a pharmaceutically-acceptable salt thereof.

The present invention provides a method for treating or preventing stress disorder, depression or anxiety disorder, comprising administering to a patient or an animal a therapeutically effective amount of 5-benzylaminosalicylic acid derivative represented by the chemical formula 1 or its pharmaceutically acceptable salts.

The present inventors have prepared and evaluated a lot of compounds, and succeeded in inventing the fact that the 5-benzylaminosalicylic acid derivatives or its pharmaceutically acceptable salts are much useful for treating or preventing stress disorder, depression or anxiety disorder as well as safe.

In the present invention, above 5-benzylaminosalicylic acid derivatives include 5-benzylaminosalicylic acid itself.

Preferably, in the chemical formula 1, alkyl (including ‘alkyl’ of haloalkyl) is C₁-C₅ alkyl, and more preferably C₁-C₃ alkyl. More specifically, preferable alkyl includes, but is not limited to, methyl, ethyl, propyl, isopropyl, butyl, sec-butyl and tert-butyl. Alkoxy (including ‘alkoxy’ of haloalkoxy), preferably, is C₁-C₅ alkoxy, and more preferably C₁-C₃ alkoxy. More specifically, preferable alkoxy includes, but is not limited to, methoxy, ethoxy, and propanoxy. Halogen includes, but is not limited to, fluoride, chloride, bromide, and iodide. Preferably, alkanoyl is C₂-C₁₀ alkanoyl, and more preferably C₃-C₅ alkanoyl. More specifically, preferable alkanoyl includes, but is not limited to, ethanoyl, propanoyl, and cyclohexanecarbonyl.

Preferable examples of the 5-benzylaminosalicylic acid derivative include, but are not limited to, 2-hydroxy-5-phenethylamino-benzoic acid (compound 1), 2-hydroxy-5-[2-(4-trifluoromethyl-phenyl)-ethylamino]-benzoic acid (compound 2), 2-hydroxy-5-[2-(3-trifluoromethyl-phenyl)-ethylamino]-benzoic acid (compound 3), 5-[2-(3,5-bis-trifluoromethyl-phenyl)-ethylamino]-2-hydroxy-benzoic acid (compound 4), 2-hydroxy-5-[2-(2-nitro-phenyl)-ethylamino]-benzoic acid (compound 5), 5-[2-(4-chloro-phenyl)-ethylamino]-2-hydroxy-benzoic acid (compound 6), 5-[2-(3,4-difluoro-phenyl)-ethylamino]-2-hydroxy-benzoic acid (compound 7), 5-[2-(3,4-dichloro-phenyl)-ethylamino]-2-hydroxy-benzoic acid (compound 8), 5-[2-(4-fluoro-2-trifluoromethyl-phenyl)-ethylamino]-2-hydroxy-benzoic acid (compound 9), 5-[2-(2-fluoro-4-trifluoromethyl-phenyl)-ethylamino]-2-hydroxy-benzoic acid (compound 10), 2-hydroxy-5-[2-(4-methoxy-phenyl)-ethylamino]-benzoic acid (compound 11), 2-hydroxy-5-(2-o-tolyl-ethylamino)-benzoic acid (compound 12), 2-hydroxy-5-(3-phenyl-propylamino)-benzoic acid (compound 13), 2-hydroxy-5-[3-(4-trifluoromethyl-phenyl)-propylamino]-benzoic acid (compound 14), 5-[3-(4-fluoro-phenyl)-propylamino]-2-hydroxy-benzoic acid (compound 15), 5-[3-(3,4-dichloro-phenyl)-propylamino]-2-hydroxy-benzoic acid (compound 16), 2-hydroxy-5-(3-p-tolyl-propylamino)-benzoic acid (compound 17), 2-acetoxy-5-[2-(4-trifluoromethyl-phenyl)-ethylamino]-benzoic acid (compound 18), and 5-[2-(2-chloro-phenyl)-ethylamino]-2-hydroxy-benzoic acid (compound 19), 5-benzylaminosalicylic acid (compound 20), 5-(4-nitrobenzyl)aminosalicylic acid (compound 21), (5-(4-chlorobenzyl)aminosalicylic acid (compound 22), (5-(4-trifluoromethylbenzyl)aminosalicylic acid (compound 23), (5-(4-fluorobenzyl)aminosalicylic acid (compound 24), 5-(4-methoxybenzyl)aminosalicylic acid (compound 25), 5-(4-pentafluorobenzyl)aminosalicylic acid (compound 26), 5-(4-nitrobenzyl)amino-2-hydroxy ethylbenzoate (compound 27), 5-(4-nitrobenzyl)-N-acetylamino-2-hydroxy ethylbenzoate (compound 28), 5-(4-nitrobenzyl)-N-acetylamino-2-acetoxy ethylbenzoate (compound 29), 5-(4-nitrobenzoyl)aminosalicylic acid (compound 30), 5-(4-nitrobenzenesulfonyl)aminosalicylic acid (compound 31), 5-[2-(4-nitrophenyl)-ethyl]aminosalicylic acid, and (compound 32) 5-[3-(4-nitrophenyl)-n-propyl]aminosalicylic acid (compound 33).

The 5-benzylaminosalicylic acid derivative or its pharmaceutically acceptable salt of the present invention can be prepared by, but is not limited to, the reaction schemes represented in U.S. Pat. No. 6,573,402.

The term “pharmaceutically acceptable salt” of the present invention means salts produced by non-toxic or little toxic acid or base. In case that the compound of the present invention is acidic, base addition salts of the compound of the present invention can be made by reacting the free base of the compound with enough amount of desirable base and adequate inert solvent. Pharmaceutically acceptable base addition salt includes, but is not limited to, sodium, potassium, calcium, ammonium, magnesium or salt made by organic amino. In case that the compound of the present invention is basic, acid addition salts of the compound of the compound can be made by reacting the free base of the compound with enough amount of desirable acid and adequate inert solvent. Pharmaceutically acceptable acid addition salt includes, but is not limited to, propionic acid, isobutylic acid, oxalic acid, malic acid, malonic acid, benzoic acid, succinic acid, suberic acid, fumaric acid, mandelic acid, phthalic acid, benzenesulfonic acid, p-tolylsulfonic acid, citric acid, tartaric acid, methanesulfonic acid, hydrochloric acid, bromic acid, nitric acid, carbonic acid, monohydrogencarbonic acid, phosphoric acid, monohydrogen-phosphoric acid, dihydrogen-phosphoric acid, sulfuric acid, monohydrogen-sulfuric acid, hydrogen iodide, and phosphorous acid. In addition, the pharmaceutically acceptable salt of the present invention includes, but is not limited to, a salt of amino acid like arginate and an analog of organic acid like glucuronic or galactunoric.

For example, a pharmaceutically acceptable salt of 2-hydroxy-5-[2-(4-trifluoromethyl-phenyl)-ethylamino]-benzoic acid (compound 2), one preferable example of the present invention, can be prepared by the below reaction scheme 1. However, the following reaction methods are offered by way of illustration and are not intended to limit the scope of the invention.

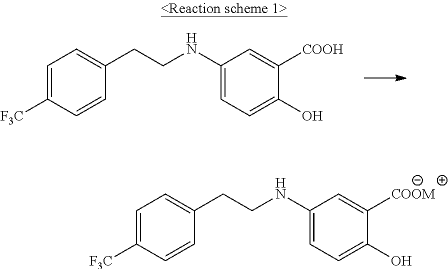

In the scheme, M is a pharmaceutically acceptable metal or basic organic compound such as diethylamine, lithium, sodium and potassium.

In more detail, diethylamine salt can be prepared by dissolving a compound in alcohol, adding dropwise diethylamine, stirring the mixture, distilling in vacuo, and crystallizing the residue by adding ether. Alkali metal salt can be made by preparing desirable salt with inorganic reagent like lithium hydroxide, sodium hydroxide, potassium hydroxide in solvent like alcohol, acetone, acetonitrile and then freeze-drying. In addition, according to the similar method, lithium salt can be made with lithium acetate, sodium salt can be made with sodium 2-ethylhexanoate or sodium acetate, and potassium salt can be made with potassium acetate.

Some of the compounds of the present invention may be hydrated form, and may exist as solvated or unsolvated form. A part of compounds according to the present invention exist as crystal form or amorphous form, and any physical form is included in the scope of the present invention. In addition, some compounds of the present invention may contain one or more asymmetric carbon atoms or double bond, and therefore exists in two or more stereoisomeric forms like racemate, enantiomer, diastereomer, geometric isomer, etc. The present invention includes these individual stereoisomers of the compounds of the present invention.

The present invention also provides a pharmaceutical composition comprising the 5-benzylaminosalicylic acid derivative represented by the above chemical formula 1 or its pharmaceutically acceptable salt; and pharmaceutically acceptable excipient or additive. The 5-benzylaminosalicylic acid derivative represented by the above chemical formula 1 or its pharmaceutically acceptable salt of the present invention may be administered alone or with any convenient carrier, diluent, etc. and a formulation for administration may be single-dose unit or multiple-dose unit.

The pharmaceutical composition of the present invention may be formulated in a solid or liquid form. The solid formulation includes, but is not limited to, a powder, a granule, a tablet, a capsule, a suppository, etc. Also, the solid formulation may further include, but is not limited to, a diluent, a flavoring agent, a binder, a preservative, a disintegrating agent, a lubricant, a filler, etc. The liquid formulation includes, but is not limited to, a solution such as water solution and propylene glycol solution, a suspension, an emulsion, etc., and may be prepared by adding suitable additives such as a coloring agent, a flavoring agent, a stabilizer, a thickener, etc.

For example, a powder can be made by simply mixing the 5-benzylaminosalicylic acid derivative of the present invention and pharmaceutically acceptable excipients like lactose, starch, microcrystalline cellulose. A granule can be prepared as follows: mixing the compound or its pharmaceutically acceptable salt, a pharmaceutically acceptable diluent and a pharmaceutically acceptable binder such as polyvinylpyrrolidone, hydroxypropylcellulose, etc; and wet-granulating with adequate solvent like water, ethanol, isopropanol, etc, or direct-compressing with compressing power. In addition, a tablet can be made by mixing the granule with a pharmaceutically acceptable lubricant such as magnesium stearate, and tabletting the mixture.

The pharmaceutical composition of the present invention may be administered in forms of, but not limited to, oral formulation, injectable formulation (for example, intramuscular, intraperitoneal, intravenous, infusion, subcutaneous, implant), inhalable, intranasal, vaginal, rectal, sublingual, transdermal, topical, etc. depending on the disorders to be treated and the patient's conditions. The composition of the present invention may be formulated in a suitable dosage unit comprising a pharmaceutically acceptable and non-toxic carrier, additive and/or vehicle, which all are generally used in the art, depending on the routes to be administered. Depot type of formulation being able to continuously release drug for desirable time also is included in the scope of the present invention.

The present invention also provides a use of the 5-benzylaminosalicylic acid derivative or its pharmaceutically acceptable salt for treating or preventing stress disorder, depression, or anxiety disorder. That is, the present invention provides a pharmaceutical composition for treating or preventing stress disorder, depression, or anxiety disorder, comprising the 5-benzylaminosalicylic acid derivative represented by the above chemical formula 1 or its pharmaceutically acceptable salt. More specifically, the 5-benzylaminosalicylic acid derivative or its pharmaceutically acceptable salt can be used for treating or preventing stress disorder such as acute stress disorder, post-traumatic stress disorder, anxiety disorder such as social anxiety disorder, generalized anxiety disorder, panic disorder, agoraphobia, substance-induced anxiety disorder, anxiety disorder due to a general medical condition, or depression.

However, the use of the 5-benzylaminosalicylic acid derivative or its pharmaceutically acceptable salt according to the present invention is not limited to the above concrete disease names.

For treating stress disorder, depression, or anxiety disorder, the compound of the present invention may be administered daily at a dose of approximately 0.01 mg/kg to approximately 100 g/kg, preferably approximately 0.1 mg/kg to approximately 10 g/kg. However, the dosage may be varied according to the patient's conditions (age, sex, body weight, etc.), the severity of patients in need thereof, the used effective components, diets, etc. The compound of the present invention may be administered once a day or several times a day in divided doses, if necessary.

### BRIEF DESCRIPTION OF THE DRAWINGS

FIG. 1 is the result evaluating the antidepressant effect of compound 2 using tail suspension test.

*, p<0.05 vs vehicle group, BID (twice a day)

FIG. 2 is the result evaluating the anti-stress effect of compound 2 using restraint stress model.

FIG. 3 is the result showing the effect of the administration to compound 2 in open field test.

*, p<0.05 vs vehicle group

FIG. 4 is the result evaluating the antidepressant effect of compound 2 using forced swimming test.

*, p<0.05 vs vehicle group

FIG. 5 is the result evaluating the antidepressant effect of compound 2 using restraint stress test.

* p<0.05 vs non-stressed vehicle (control 1) group

# p<0.05 vs stressed vehicle (control 2) group

FIG. 6 is the result evaluating the anxiolytic effect of compound 2 using stress-induced anxiety.

* p<0.05 vs non-stressed vehicle (control 1) group

# p<0.05 vs stressed vehicle (control 2) group

### EXAMPLES

Hereinafter, the present invention is described in considerable detail to help those skilled in the art understand the present invention. However, the following examples are offered by way of illustration and are not intended to limit the scope of the invention. It is apparent that various changes may be made without departing from the spirit and scope of the invention or sacrificing all of its material advantages.

### Example 1

### Evaluation of Antidepressant Effect of Compound 2 on Tail Suspension Test (TST)

The effectiveness of the compound was evaluated using a tail-suspension test as in vivo method for depression. In this test immobility time of animals typically for 5-10 minutes is recorded using a manual or an automated device. The mouse suspended by the tail shows alternate activity (movement) and immobility and drugs with antidepressant action commonly reduce the immobility time in this test.

Male ICR mice weighing 28-30 g of body weight were used throughout the study. Experimental animals were kept in an animal breeding room with a 12 h dark/12 h light cycle and accommodated by 8 mice per cage supplied freely with water and feeding. The mice consisted of 8 animals per group were randomly allocated to the treatment groups.

Compound 2 was suspended in 10% lutrol solution and the all suspended solutions were administered orally to the mice twice daily at a volume of 4 ml/kg. The control group was treated only with 10% lutrol solution in the same manner. All the treatment of drugs or vehicle to the animals were given 1 hour before the test (n=8/group).

1. Control 1 (vehicle, 10% lutrol)

2. Compound 21.25 mg/kg (oral administration, twice daily)

3. Compound 22.5 mg/kg (oral administration, twice daily)

4. Compound 25 mg/kg (oral administration, twice daily)

5. Compound 212.5 mg/kg (oral administration, twice daily)

6. Compound 225 mg/kg (oral administration, twice daily)

7. Compound 250 mg/kg (oral administration, twice daily)

This test was evaluated according to the method described in a previous publication [Psycopharmacology 85, 367-370, Steru et al. (1985)]. At the day of experiment, the mice were acclimatized in the testing room over 60 minutes and given with the compound, and then the test was conducted. After 1 h after drug treatment, the mice were taped with experimental adhesive tape, at approximately 1 cm from the tip of the tail. The mice were individually suspended on an experimental shelf about 58 cm above a table top by attaching the adhesive tape at the tip of the tail to the experimental shelf. And after that, total immobility time was measured for 6 min in the absence of noise. The total immobility time was measured during the final 4 min of a 6 min test session and the data were expressed as mean and analyzed by one-way ANOVA followed by Tukey's HSD (Honestly Significant Difference) post hoc comparison.

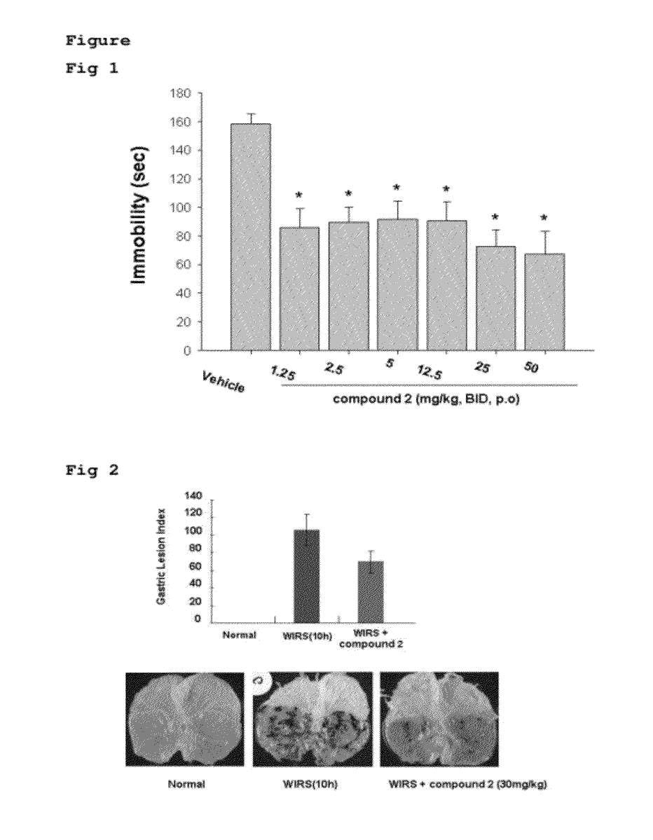

As shown in FIG. 1, all the groups treated with compound 2 decreased immobility time during experimental time of 4 minutes. In groups treated with compound 2 at doses of 1.25 mg/kg, 2.5 mg/kg, 5 mg/kg, 12.5 mg/kg, 25 mg/kg, and 50 mg/kg, the total immobility time were significantly decreased by 46%, 43%, 42%, 38%, 54%, and 57%, respectively, compared with the control group. Thus, it can be known that compound 2 according to the present invention has antidepressant activity.

### Example 2

### The Effect of Compound 2 in Stress Models 2-1. Water Immersion Restraint Stress (WIRS) Induction

The Sprague-Dawley (SD) rats weighing 200 g of body weight were deprived of food for over 24 hours and then were given with the experimental compound. After 1 hour, the rats were placed in a stress cage and immersed into water at 24° C. for 10 hours to induce WIRS

### 2-2. The Effect of Compound 2 on Gastric Damage in Stress Model

The SD rats weighing 200 g of body weight were deprived of food for 24 h and then were given with 30 mg/kg of compound 2. After 1 hour, the rats were placed in a stress cage and immersed into water for 10 hours. After WIRS for 10 hours, the rats were sacrificed under anesthesia with ether. Each stomach was isolated, soaked and fixed in 10 ml of 2% formalin solution for 10 minutes, and then opened along the greater curvature of the stomach. After spreading the stomach, stress-induced hemorrhagic lesions were examined macroscopically, and the results were shown in FIG. 2.

As shown in FIG. 2, treatment with 30 mg/kg of compound 2 definitely reduced WIRS-induced gastric lesion index.

### Example 3

### The Effect of Compound 2 on Locomotor Activity Using Open Field Test

The locomotor activity using open field test measured to know influence of drug treatment on activity in animals. Male ICR mice weighing 20-23 g of body weight were used throughout the study.

Experimental animals were kept in an animal breeding room with a h dark/12 h light cycle and accommodated by 8 mice per cage supplied freely with water and feeding. The mice consisted of 8 animals per group were randomly allocated to the treatment groups.

Compound 2 was suspended in 10% lutrol solution and all the suspended solutions were administered orally to the mice twice daily at a volume of 5 ml/kg. The control group was treated only with 10% lutrol solution in the same manner. All the treatment of drugs or vehicle to the animals were given 1 hour before the test (n=8/group).

1. Control 1 (vehicle, 10% lutrol)

2. Compound 21.25 mg/kg (oral administration, twice daily)

3. Compound 22.5 mg/kg (oral administration, twice daily)

4. Compound 25 mg/kg (oral administration, twice daily)

5. Compound 212.5 mg/kg (oral administration, twice daily)

6. Compound 225 mg/kg (oral administration, twice daily)

7. Compound 250 mg/kg (oral administration, twice daily)

The open field was consisted of square arena (58 cm×58 cm) surrounded by a 30 cm height wall. The floor of the arena was divided into 12 equal squares. At the start of each trial, a mouse was placed in a corner of the field and was allowed to freely move the arena and the moving trajectory for 5 min was measured. The behavior of animals was recorded and analyzed using a videotracking software (Etho Vision 3.0, Noldus Information Technology, Leesburg, Va.) attached to the ceiling above the middle of the arena.

The collected data were analyzed by one-way ANOVA followed by Tukey's HSD (Honestly Significant Difference) post hoc comparison.

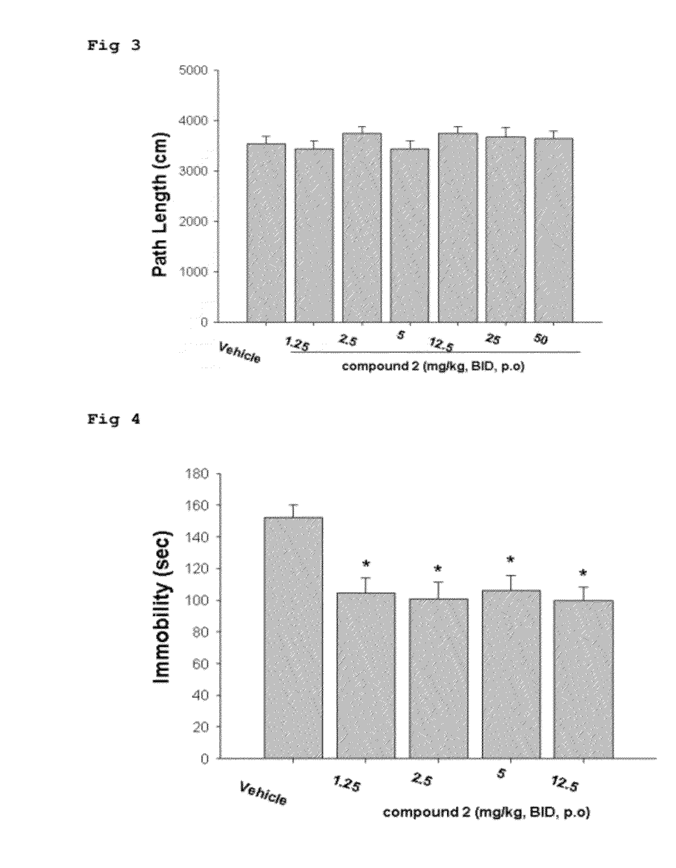

As shown in FIG. 3, locomotor activity was not different among groups, suggesting that compound 2 shows antidepressant activity without influencing on the locomotor activity.

### Example 4

### Evaluation of Antidepressant Effect on Forced Swimming Test

Forced swimming test is used to select compounds with therapeutic efficacy for depression. An Animal located in a cylinder filled with water shows various avoidance behaviors or immobility. The immobility time is counted whenever the animal remained floating passively in the water without struggling except minimal movement to protrude its head above the water surface. Antidepressants significantly improve the avoidance behavior or immobility.

Nine-week-old male C57BL/6 mice weighing 20-23 g of body weight were used through this study. Experimental animals were kept in an animal breeding room with a 12 h dark/12 h light cycle and accommodated by 8 mice per cage supplied freely with water and feeding. The mice consisted of 8 animals per group were randomly allocated to the treatment groups.

Compound 2 was suspended in 10% lutrol solution and all the suspended solutions were administered orally to the mice twice daily at a volume of 5 ml/kg. The control group was treated only with 10% lutrol solution in the same manner. All the treatment of drugs or vehicle to the animals were given 1 hour before the test (n=8/group).

1. Control 1 (vehicle, 10% lutrol)

2. Compound 21.25 mg/kg (oral administration, twice daily (BID))

3. Compound 22.5 mg/kg (oral administration, twice daily (BID))

4. Compound 25 mg/kg (oral administration, twice daily (BID))

5. Compound 212.5 mg/kg (oral administration, twice daily (BID))

At the day of the experiment, the mice were acclimatized in the testing room over 60 minutes, orally given with the compound 2 or vehicle control, and received the forced swimming test. For the forced swimming test, a mouse was individually forced to swim inside an open glass cylindrical bath with height of 26 cm and diameter of 14 cm filled with water at 24±1° C. up to 16 cm high from the bottom so that the mouse tail could not reach the bottom. One hour after the mouse were orally given compound 2 and control vehicle, then the mice were exposed to forced swimming for 6 min in the water bath and their movement was recorded using a video camera system. The total duration of immobility was measured as a mean during the final 4 min of a 6-min test session and the data were statistically analyzed with one-way ANOVA followed by Tukey's HSD (Honestly Significant Difference) post hoc comparison.

The results were represented in FIG. 4. All the groups treated with compound 2 decreased immobility time during the test time of 4 minutes. Immobility time in the groups treated with compound 2 was significantly decreased compared with the vehicle control group, showing 31%, 34%, 30%, and 34% at doses of 1.25 mg/kg, 2.5 mg/kg, 5 mg/kg, and 12.5 mg/kg, respectively.

### Example 5

### Evaluation of Antidepressant Effect on Repeated Restraint Stress Test

Currently, two aspects of depression, efficacy of antidepressant and response on stress, are mainly utilized for the depression studies using experimental animals.

In case of a realistic animal model of depression, in which symptoms corresponding to depression are created by exposing animals to a similar situation to be able to evoke corresponding human diseases, a study on the physiological defects becomes possible, which reside as causes in the diseases. Such realistic animal models of depression evoke depression to animals mainly by adding stress such as isolation, electric shock, forced swimming, environmental changes, and temperature changes and so on.

To test the efficacy of compounds on a stress-induced depression a restraint stress animal model was used. After evoking depression symptoms in a mouse corresponding to a human depression by exposing a stressed condition that it was placed in a narrow space to be unable to move, whether the compounds have any anti-depression effects were tested after treatment to this animal.

Nine-week-old male C57BL/6 mice weighing 20-23 g of body weight were used through this study. Experimental animals were kept in an animal breeding room with a 12 h dark/12 h light cycle and accommodated by 5-6 mice per cage supplied freely with water and feeding. The mice consisted of 11 animals per group were randomly allocated to the treatment groups.

Compound 2 was suspended in 10% lutrol solution and all the suspended solutions were administered orally to the mice twice daily at a volume of 5 ml/kg. The control group was treated only with 10% lutrol solution in the same manner. All the treatment of drugs or vehicle to the animals were given 1 hour before the test (n=11/group).

1. Control 1 (vehicle, 10% lutrol)

2. Compound 22.5 mg/kg (oral administration, twice daily (BID))

3. Compound 25 mg/kg (oral administration, twice daily (BID))

4. Control 2 (10% lutrol)+restraint stress

5. Compound 22.5 mg/kg (oral administration, twice daily (BID))+restraint stress

6. Compound 25 mg/kg (oral administration, twice daily (BID))+restraint stress

Animals were subjected to restraint-stress one hour after compound 2 or 10% lutrol was administered orally. Mice were exposed to restraint stress everyday 2 hr for 14 days and received a forced swim test such as <Example 4> as an index for depression.

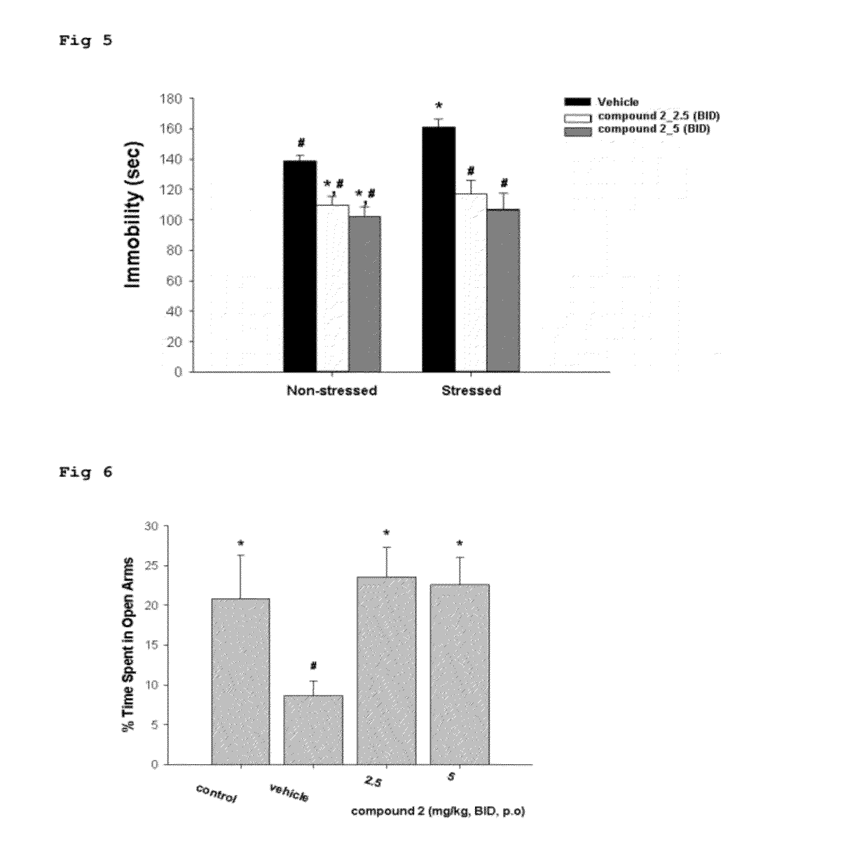

The results were shown in FIG. 5. The immobility time was significantly increased by 16% in the stressed control group which received stress for 2 weeks (control 2) compared with unstressed control group (control 1) However, immobility time in all the groups treated with compound 2 was significantly decreased at doses of 2.5, mg/kg and 5 mg/kg by 32% and 39%, respectively, compared with stressed control group (control 2)

In addition, to determine the effect of long-term treatment of compound 2 on depression, a forced swimming test was performed after administration of compound 2 for 2 weeks. Immobility time was significantly decreased in groups treated with compound 2 at doses of 2.5 mg/kg and 5 mg/kg by 21% and 26%, respectively, compared with unstressed group (control 1).

### Example 6

### Anxiolytic Effect of Compound 2 on Stress-Induced Anxiety

To evaluate the effect of compound 2 on stress-induced anxiety, we used a restraint stress animal model. Anxiety was evoked by exposing stress that a mice was restrained to be immovable in a narrow space, and whether the compound could show anxiolytic effect after administration the animals.

When experimental animals are exposed to stress, various physiological changes occur, which can be measured through several behavioral tests. In this study, anxiety of experimental animals was evaluated by an elevated plus maze (EPM). EPM apparatus consisted of two open arms, and two closed arms which are horizontally crossed. It is designed that the cross path is located 60 cm above the floor for the animals to feel anxiety.

Nine-week-old male C57BL/6 mice weighing 20-23 g of body weight were used through this study. Experimental animals were kept in an animal breeding room with a 12 h dark/12 h light cycle and accommodated by 8 mice per cage supplied freely with water and feeding. The mice consisted of 16 animals per group were randomly allocated to the treatment groups.

Compound 2 was suspended in 10% lutrol solution and all the suspended solutions were administered orally to the mice at a volume of 5 ml/kg. The control group was treated only with 10% lutrol solution in the same manner. All the treatment of drugs or vehicle to the animals were given 1 hour before the test (n=16/group).

1. Control 1 (10% lutrol)

2. Control 2 (10% lutrol)+restraint stress

3. Compound 22.5 mg/kg (oral administration, twice daily (BID))+restraint stress

4. Compound 25 mg/kg (oral administration, twice daily (BID))+restraint stress

Animals were subjected to restraint-stress one hour after compound 2 or 10% lutrol was administered orally. Mice were exposed to restraint stress everyday 2 hr for 14 days and received an elevated plus maze test as an index for anxiety.

At the day of experiment, animals exposed to restraint stress after animals were acclimatized to testing room over 60 minutes, and then were orally administered with the compound. One hour after administration the oral administration of compound 2 or lutrol, experimental animals were placed at the center of EPM and the animal behaviors were recorded during total 5 minutes using a camera mounted on the ceiling and a computer program (etho vision 3.0 (Noldus Information Technology)). The collected data were expressed as % time spent in open arms and the statistical analysis was done using one-way ANOVA followed by LSD (Least significant difference) post hoc comparisons.

The results were shown in FIG. 6. When % time spent in open arm was observed, the rate showed 21% and 9% in unstressed (control 1) and stressed (control 2) groups, respectively, suggesting that anxiety was induced by current chronic stress. On the other hand, % time spent in open arm was significantly increased in the group treated with compound 2 at doses of 2.5 mg/kg and 5 mg/kg by 24% and 23%, respectively, compared with the stressed group (control 2), showing similar levels to the unstressed group (control 1).

### INDUSTRIAL APPLICABILITY

The present invention provide a pharmaceutical composition useful for treating or preventing stress disorder, depression, or anxiety disorder, comprising the 5-benzylaminosalicylic acid derivative represented by the chemical formula 1 or its pharmaceutically acceptable salt, and a method for treating or preventing stress disorder, depression, or anxiety disorder, using the pharmaceutical composition. The pharmaceutical composition of the present invention is very useful for treating or preventing stress disorder, depression, or anxiety disorder, as well as safe.

1. A pharmaceutical composition comprising 5-benzylaminosalicylic acid derivative represented by the chemical formula 1 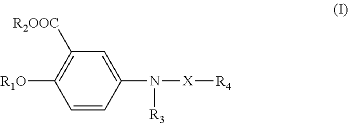
wherein,
X is CO, SO₂ or (CH₂)ₙ (where n is an integer of 1 to 5, inclusive);
R₁ is hydrogen, alkyl or alkanoyl;
R₂ is hydrogen or alkyl;
R₃ is hydrogen or an acetyl group; and
R₄ is phenyl group which is unsubstituted or substituted with one or more of the group consisting of nitro, halogen, haloalkyl, and C₁-C₅ alkoxy;
or a pharmaceutically-acceptable salt thereof.

2. The pharmaceutical composition of claim 1, wherein the 5-benzylaminosalicylic acid derivative is any one selected from the group consisting of 2-hydroxy-5-phenethylamino-benzoic acid, 2-hydroxy-5-[2-(4-trifluoromethyl-phenyl)-ethylamino]benzoic acid, 2-hydroxy-5-[2-(3-trifluoromethyl-phenyl)-ethylamino]-benzoic acid, 5-[2-(3,5-bis-trifluoromethyl-phenyl)-ethylamino]-2-hydroxy-benzoic acid, 2-hydroxy-5-[2-(2-nitro-phenyl)-ethylamino]-benzoic acid, 5-[2-(4-chloro-phenyl)-ethylamino]-2-hydroxy-benzoic acid, 5-[2-(3,4-difluoro-phenyl)-ethylamino]-2-hydroxy-benzoic acid, 5-[2-(3,4-dichloro-phenyl)-ethylamino]-2-hydroxy-benzoic acid, 5-[2-(4-fluoro-2-trifluoromethyl-phenyl)-ethylamino]-2-hydroxy-benzoic acid, 5-[2-(2-fluoro-4-trifluoromethyl-phenyl)-ethylamino]-2-hydroxy-benzoic acid, 2-hydroxy-5-[2-(4-methoxy-phenyl)-ethylamino]-benzoic acid, 2-hydroxy-5-(2-o-tolyl-ethylamino)-benzoic acid, 2-hydroxy-5-(3-phenyl-propylamino)-benzoic acid, 2-hydroxy-5-[3-(4-trifluoromethyl-phenyl)-propylamino]-benzoic acid, 5-[3-(4-fluoro-phenyl)-propylamino-]-2-hydroxy-benzoic acid, 5-[3-(3,4-dichloro-phenyl)-propylamino]-2-hydroxy-benzoic acid, 2-hydroxy-5-(3-p-tolyl-propylamino)-benzoic acid, 2-acetoxy-5-[2-(4-trifluoromethyl-phenyl)-ethylamino]-benzoic acid, and 5-[2-(2-chloro-phenyl)-ethylamino]-2-hydroxy-benzoic acid, 5-benzylaminosalicylic acid, 5-(4-nitrobenzyl)aminosalicylic acid, (5-(4-chlorobenzyl)aminosalicylic acid, (5-(4-trifluoromethylbenzyl)aminosalicylic acid, (5-(4-fluorobenzyl)aminosalicylic acid, 5-(4-methoxybenzyl)aminosalicylic acid, 5-(4-pentafluorobenzyl)aminosalicylic acid, 5-(4-nitrobenzyl)amino-2-hydroxy ethylbenzoate, 5-(4-nitrobenzyl)-N-acetylamino-2-hydroxy ethylbenzoate, 5-(4-nitrobenzyl)-N-acetylamino-2-acetoxy ethylbenzoate, 5-(4-nitrobenzoyl)aminosalicylic acid, 5-(4-nitrobenzenesulfonyl)aminosalicylic acid, 5-[2-(4-nitrophenyl)-ethyl]aminosalicylic acid, and 5-[3-(4-nitrophenyl)-n-propyl]aminosalicylic acid.

3. The pharmaceutical composition of claim 2, wherein the 5-benzylaminosalicylic acid derivative is 2-hydroxy-5-[2-(4-trifluoromethyl-phenyl)-ethylamino]-benzoic acid.

4. (canceled)

5. (canceled)

6. A method for treating or preventing stress disorder, depression, or anxiety disorder, comprising administering to a subject in need thereof a therapeutically effective amount of the 5-benzylaminosalicylic acid derivative represented by the chemical formula 1: 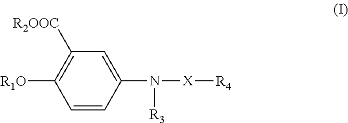
wherein,
X is CO, SO₂ or (CH₂)ₙ (where n is an integer of 1 to 5, inclusive);
R₁ is hydrogen, alkyl or alkanoyl;
R₂ is hydrogen or alkyl;
R₃ is hydrogen or an acetyl group; and
R₄ is phenyl group which is unsubstituted or substituted with one or more of the group consisting of nitro, halogen, haloalkyl, and C₁-C₅ alkoxy;
or a pharmaceutically-acceptable salt thereof.

7. The method of claim 6, wherein the 5-benzylaminosalicylic acid derivative is any one selected from the group consisting of 2-hydroxy-5-phenethylamino-benzoic acid, 2-hydroxy-5-[2-(4-trifluoromethyl-phenyl)-ethylamino]-benzoic acid, 2-hydroxy-5-[2-(3-trifluoromethyl-phenyl)-ethylamino]-benzoic acid, 5-[2-(3,5-bis-trifluoromethyl-phenyl)-ethylamino]-2-hydroxy-benzoic acid, 2-hydroxy-5-[2-(2-nitro-phenyl)-ethylamino]-benzoic acid, 5-[2-(4-chloro-phenyl)-ethylamino]-2-hydroxy-benzoic acid, 5-[2-(3,4-difluoro-phenyl)-ethylamino]-2-hydroxy-benzoic acid, 5-[2-(3,4-dichloro-phenyl)-ethylamino]-2-hydroxy-benzoic acid, 5-[2-(4-fluoro-2-trifluoromethyl-phenyl)-ethylamino]-2-hydroxy-benzoic acid, 5-[2-(2-fluoro-4-trifluoromethyl-phenyl)-ethylamino]-2-hydroxy-benzoic acid, 2-hydroxy-5-[2-(4-methoxy-phenyl)-ethylamino]-benzoic acid, 2-hydroxy-5-(2-o-tolyl-ethylamino)-benzoic acid, 2-hydroxy-5-(3-phenyl-propylamino)-benzoic acid, 2-hydroxy-5,3-(4-trifluoromethyl-phenyl)-propylamino)-benzoic acid, 5-[3-(4-fluoro-phenyl)-propylamino]-2-hydroxy-benzoic acid, 5-[3-(3,4-dichloro-phenyl)-propylamino]-2-hydroxy-benzoic acid, 2-hydroxy-5-(3-p-tolyl-propylamino)-benzoic acid, 2-acetoxy-5-[2-(4-trifluoromethyl-phenyl)-ethylamino]-benzoic acid, and 5-[2-(2-chloro-phenyl)-ethylamino]-2-hydroxy-benzoic acid, 5-benzylaminosalicylic acid, 5-(4-nitrobenzyl)aminosalicylic acid, (5-(4-chlorobenzyl)aminosalicylic acid, (5-(4-trifluoromethylbenzyl)aminosalicylic acid, (5-(4-fluorobenzyl)aminosalicylic acid, 5-(4-methoxybenzyl)aminosalicylic acid, 5-(4-pentafluorobenzyl)aminosalicylic acid, 5-(4-nitrobenzyl)amino-2-hydroxy ethylbenzoate, 5-(4-nitrobenzyl)-N-acetylamino-2-hydroxy ethylbenzoate, 5-(4-nitrobenzyl)-N-acetylamino-2-acetoxy ethylbenzoate, 5-(4-nitrobenzoyl)aminosalicylic acid, 5-(4-nitrobenzenesulfonyl)aminosalicylic acid, 5-[2-(4-nitrophenyl)-ethyl]aminosalicylic acid, and 5-[3-(4-nitrophenyl)-n-propyl]aminosalicylic acid.

8. The method of claim 7, wherein the 5-benzylaminosalicylic acid derivative is 2-hydroxy-5-[2-(4-trifluoromethyl-phenyl)-ethylamino]-benzoic acid.

9. The method of claim 6, wherein the stress disorder is any one selected from the group consisting of acute stress disorder, and post-traumatic stress disorder.

10. The method of claim 6, wherein the anxiety disorder is any one selected from the group consisting of social anxiety disorder, generalized anxiety disorder, panic disorder, agoraphobia, substance-induced anxiety disorder, and anxiety disorder due to a general medical condition.
